(19) United States
(12) Patent Application Publication
(10) Pub. No.: US 2022/0238222 A1
(43) Pub. Date: Jul. 28, 2022

(54) REMOTE HEALTH MONITORING SYSTEM AND METHOD FOR HOSPITALS AND CITIES

(71) Applicant: Jérémie NEUBERG, Paris (FR)

(72) Inventor: Jérémie NEUBERG, Paris (FR)

(21) Appl. No.: 17/611,733

(22) PCT Filed: May 18, 2020
(86) PCT No.: PCT/EP2020/063870

§ 371 Date: Nov. 16, 2021

(30) Foreign Application Priority Data
May 16, 2019 (FR) 1905164

(51) Int. Cl. G16H 40/67; G16H 70/00; G16H 50/30; G16H 10/20

(52) U.S. Cl. CPC G16H 40/67; G16H 10/20; G16H 50/30; G16H 70/00

(57) ABSTRACT

A telehealth system for rescuing a user, which includes a central server, a transmission system, and peripheral device(s) for transmitting health-related user's data, including physiological parameter measurement(s), to the central server via the transmission system. The central server includes: a module receiving the health-related user's data; a memory for storing: the data received, a user's alert list, an instructions library, a physiological parameters library, and reference values/ranges for physiological parameters; and a processing unit for: comparing the physiological parameter measurement(s) with reference values/ranges in the physiological parameters library; determining at least one risk index from the comparison; determining a low, moderate, or high level for the risk index, and executing instruction(s) from the instruction list, including, for moderate or high risk levels, automatically sending a communication request to the user's alert list and, for no response, calling an emergency service or ordering an assistance service to fetch the user.

### FIELD OF INVENTION

The present invention pertains to the field of telemedicine, more precisely it relates to a telehealth system and method for healthcare facilities, individuals, and communities.

### BACKGROUND OF INVENTION

The present invention relates to a telemedicine system, more precisely for remote monitoring of a user, preferentially a patient suffering from a pathology.

The system according to the present invention comprises a plurality of algorithms and connected medical devices configured to continuously or intermittently transmit data.

The patent application WO2016126859 is an example of a system and a method of the prior art for displaying a summary of patient records obtained through various database systems for patient admission and treatment in different scenarios.

The patent application WO2017007461 relates to a system for automated medical decision-making that includes a first parser configured to parse text associated with medical information sources to obtain medical information and a second parser configured to parse patient data to obtain processed patient data.

In these systems of the prior art the patients do not actively participate in their care. Moreover, these systems do not aim at continuously improving the healthcare workflow, neither they aim at digitalizing patients' data related to the whole patient care pathway.

The present invention aims at overcoming the problems associated with the systems of the prior art. In particular, the present invention aims at delivering a patient-centered care in which the patient is a subject playing an active role in his/her care.

### BRIEF DESCRIPTION OF THE INVENTION

The present invention relates to a telehealth system for rescuing a user in need thereof, the system comprising a central server, a transmission system and at least one peripheral device configured to continuously or intermittently transmit health-related user's data to the central server via the transmission system; the health-related user's data comprising at least one measurement of a physiological parameter; and the central server comprising:
a module configured to receive the health-related user's data;
a memory configured to store:
   the health-related user's data;
   a user's alert list comprising a plurality of recipient devices;
   a library of instructions comprising: sending a communication request to all the recipient devices in the user's alert list; triggering a call to an emergency service, ordering an assistance service to fetch the user; and
   a library of physiological parameters wherein a reference value or a reference range of values is associated with each physiological parameter;
a processing unit configured to:
   compare the at least one measurement of a physiological parameter with the reference value or the reference range of values associated with said physiological parameter in the library;
   determine at least one risk index based on the comparison;
   determine a level for the at least one risk index, the risk index level being selected from a group consisting of: low, moderate, high;
   based on the risk index level, execute at least one instruction from the list of instructions, and for a moderate or a high risk level, the at least one instruction comprises automatically sending a communication request to all the recipient devices in the user's alert list and, in case of no response within a predetermined interval of time Δt, a call to an emergency service or an order to an assistance service to fetch the user.

The call to an emergency service or an order to an assistance service may comprise the user's identification data and geo-localization data, such as for example geo-localization data obtained via user's authorizing, via Bluetooth, via triangulation, via WIFI network.

In one embodiment of the present invention, the plurality of recipient devices in the user's alert list comprises:
at least one user-side communication device;
at least one communication device of a second subject designated by the user; and/or
the communication devices that match a vicinity criterion.

In one embodiment, the vicinity criterion is being connected to the same wireless network as the at least one user's peripheral device or the at least one user-side communication device.

In one embodiment, the vicinity criterion is being located within a predetermined distance from the user-side communication device at least one peripheral device, preferentially the predetermined distance is ranging between 0 meters and 500 meters.

In a preferred embodiment, the predetermined distance is ranging between 1 meter and 100 meters.

In one embodiment, the health-related user's data further comprise:
an electronic medical record of the user and/or
the user's answers to the questions of a questionnaire, the questionnaire comprising questions that are neutral to the user and questions that are generated by means of an algorithm based on the health-related user's data;

and wherein the determination of at least one risk index is further based on the user's data in the electronic medical record and/or the user's answers to the questions of the questionnaire.

In one embodiment, the electronic medical record comprises genetic factors and/or epidemiologic factors.

In one embodiment of the present invention, the risk index is selected from a group comprising: anaphylactic shock, stroke, heart failure, cardiovascular diseases, respiratory deficiency, sleep apnea, endocrinal disease, metabolic disease, infectious or parasitic diseases, digestive diseases, immune system diseases, nephrological disorders, sexually transmitted diseases, viral diseases, smoking addiction, drug use addiction, arterial hypertension, cancer, kidney diseases, gynecological diseases, neurodegenerative diseases, neurological diseases, psychiatric diseases, post-operative risk, circulatory system disease, sudden cardiac death, nutritional diseases or a risk of sudden death.

In one embodiment, the physiological parameter is selected from a group comprising: oxygen saturation, respiratory rate, blood pressure, heart rate, temperature, weight, blood pressure, respiratory rate, blood oxygen saturation, pulse rate, heart rate variability, stroke volume, cardiac output, cardiac index, pulse pressure, systemic vascular resistance, ECG, mean arterial pressure, sweat level, skin temperature, body temperature, glucose level.

In one embodiment, the processing unit is further configured to:
in case of response to the communication request, classify said response as a rescue request response or a non-rescue request response; and
   for a rescue-request response, automatically trigger a call to an emergency service or an order to an ambulance service to fetch the user;
   for a non-rescue-request response, automatically send a second communication request to all the recipients in the user's alert list.

In one embodiment, the predetermined interval of time Δt ranges between 10 and 60 seconds. The predetermined interval of time Δt for a moderate risk index level is superior to the interval of time Δt for a high risk index level.

In one embodiment of the present telehealth system, the list of instructions further comprises:
sending an alert to a subset of devices selected from the user's alert list;
generating a questionnaire;
generating a medicine prescription;
generating a medicine purchase order for the prescribed medicine;
automatically delivering the prescribed medicine;
generating a pre-medical evaluation report;
generating a post-medical evaluation report;
scheduling a user's consultation with a healthcare provider;
sending a medication reminder to the user;
sending an alert to a healthcare provider.

The present invention also relates to a computer-implemented method for rescuing a user in need thereof, the method comprising the following steps:
a) measuring and/or collecting health-related data of a user via at least one peripheral device, the health-related data comprising at least one measurement of a physiological parameter;
b) transmitting the health-related data from the peripheral device to a central server via a transmission system, the central server comprising a memory;
c) storing, preferentially using a distributed ledger or blockchain technology, the health-related data in the memory of the central server;
d) generating a user's alert list comprising a plurality of recipient devices;
e) continuously or intermittently comparing the at least one measurement of a physiological parameter with a reference value or a reference range of values for said physiological parameter;
f) based on the result of the comparison step d) and on the user's health-related data, determining at least one risk index;
g) determining a level for the at least one risk index, the risk index level being selected from a group consisting of: low, moderate, high; based on the at least one risk index and on the risk index level, selecting at least one instruction from a library of instructions;

wherein, for a moderate or high risk, the selected at least one instruction comprises automatically sending a communication request to all the recipient devices in the user's alert list and, in case of no response to the communication request within a predetermined interval of time Δt, a call to an emergency service or an order to an assistance service to fetch the user at a time.

In one embodiment, the method of the present invention further comprises a step of
b.2) evaluating a quality metrics of the at least one measurement of a physiological parameter by means of an artifact detection algorithm;

and the step f) of determining at least one risk index is further based on the evaluated quality metrics.

In one embodiment, the step of generating a user's alert list comprising a plurality of recipient devices comprises the following steps:
receiving as input information regarding at least one user-side communication device and at least one communication device of a second subject designated by the user;
identifying neighbor devices based on a user's vicinity criterion; the preferred vicinity criterion being: identifying the communicating devices that are connected to the same wireless network as the at least one user's peripheral device or the at least one user-side communication device.
automatically generating a user's alert list comprising, as recipients: the at least one user-side communication device; the at least one communication device of a second subject designated by the user and the identified neighbor devices.

In one embodiment of the present, the method further comprises:
repeating the steps a) to c) so as to obtain health-related data from a plurality of users;
building, by means of a machine learning algorithm, a predictive model based on the data from a plurality of users.

In one embodiment, the present method further comprises a step of inputting the health-related data of a user into the predictive model and obtaining as output a value predictive of a future clinical event of the user.

The present invention further relates to a computer program product for rescuing a user in need thereof, the computer program product comprising instructions which, when the program is executed by a computer, cause the computer to carry out the steps of the method described hereabove.

The present invention also relates to a computer-readable storage medium comprising instructions which, when the program is executed by a computer, cause the computer to carry out the steps of the method described hereabove.

### DETAILED DESCRIPTION OF THE INVENTION

The present invention relates to a telehealth system, more precisely for remote monitoring and rescuing of a user, preferentially of a patient suffering from a pathology.

The system according to the present invention comprises a plurality of algorithms and connected medical devices configured to continuously or intermittently transmit data.

The aim of the present invention is to continuously improve and optimize the patient care pathways; to optimize the workflow of tasks performed by the healthcare professionals; to ease the communication between healthcare professionals; and to optimize the organization of healthcare facilities.

The present system maximizes the amount of a user's digital data that are available for diagnostic and therapeutic purpose; moreover, it automatizes the storage of said data with the twofold aim of improving the organization of healthcare facilities, such as hospitals and clinics, and implementing personalized and predictive medicine.

The purpose of the present invention is to deliver a patient-centered care, to ensure the best quality of care, to ensure that all the steps in the healthcare workflows fit together, especially the most critical steps that involve interactions between patients, healthcare professionals, general practitioners and healthcare facilities in order to meet the need of a community-based care.

The present invention is a telehealth system that allows the user play an active role throughout his/her care pathway.

The system according to the present invention comprises a secure access to data, several algorithms and a plurality of connected medical devices, watches, patches, wearables, scales or other connected objects, which are capable of transmitting data of a user at a given moment of time or continuously. The combination of these elements allows the telehealth system to perform a plurality of functions, such as for example those illustrated in FIG. 1.

The method of the present invention comprises the collection of health-related data; said data may be transmitted in the context of a remote monitoring session or a teleconsultation; submitted by a user through medical questionnaires or quality-of-life questionnaires; transmitted by connected medical devices.

The present invention aims at solving a problem commonly faced by healthcare professional or institutions, that is the impossibility to fully appreciate, interpret and follow the evolution over time of the health states of a patient at a distant location, said health states comprise the patient's mental and emotional states; the symptoms and/or the side effects related to an illness or a treatment.

Furthermore, the present invention allows to store a user's data, such as the data collected by one or more connected objects; data obtained by means of machine learning algorithms; information submitted by a user, such as symptoms; physiological parameters, such as heart rates or other health indicators. Said user's data are analyzed while taking into account information submitted by the user, such as the answers to the questions of medical questionnaires relating to symptoms, to quality-of-life and to side effects and/or while taking into account information collected from the user, such as the duration of his connections, the searches he carried out, the communications between the user and the healthcare professionals. This analysis provides models aiming at improving medical research; understanding the evolution of a disease; and optimizing the treatment choice and prescription for each patient. Therefore, this system would allow to conduct real-life or real-world clinical studies, without time limit. In addition, the results of the user's data analysis can be used for building predictive models and/or for precision medicine.

In the telehealth system according to the present invention, the questions of the questionnaires are provided by health professionals and/or are generated by means of algorithms These algorithms that generate questions can do so on the basis of the parameters received from a user and/or they cross-reference said received parameters with other data, and generate questions based on the result of the cross-referencing. The cross-referencing may comprise: comparing different received parameters among them, comparing one or more received parameters with data stored in a database in the memory of the telehealth system, comparing one or more received parameters with user's answers to previous questions.

The questionnaires can differ from one patient to another depending on individual factors such as: health state, pathology, stage of a disease, treatment, age, sex or other individual factors.

In addition, the present system allows a patient to submit further questionnaires which he has answered to, such as pre-admission and/or post-admission questionnaires.

In the present telehealth system, the aforementioned data comparison allows to generate different types of alerts: the study of a patient's case by a doctor or another healthcare professional; making a voice call, or launching a videoconference, to a patient's device by a doctor or another healthcare professional; automatically dispatching an ambulance, automatically dispatching a motorized convoy.

Said ambulance and/or motorized convoy may belong to an institution that utilizes the present telehealth system or to one of its partners.

The dispatched ambulance service examines a patient and/or transport the patient by land, sea or air, for treatment or monitoring in a hospital, a clinic, a nursing home or other place of care delivery.

In addition, the system allows to predict different medical events, such as the evolution of a pathological condition known to the user, or predict the evolution of a novel pathological condition that may occur.

The connected devices are connected with a WIFI network and/or any other data transmission system to the server on which the telehealth system is hosted.

The connected devices are visible in real time, or in a history format, in the healthcare professional-side of the system of the present invention; and/or on the editor-side, i.e. to the publisher of the connected devices; and/or in any other digital medium.

The cross-referencing of data for predictive medicine purposes can also comprise comparing data related to the family relationships of a patient in order to draw conclusions on the possible appearance of symptoms. or pathologies from one family member to another, said data related to family relationships may be submitted by the patients themselves and/or by the healthcare professionals.

The present system also comprises features related to the practical organization of the daily life of a patient, said features comprise for instance: the organization and update of the patient's medical record by various professionals with whom said medical record is shared; the personalized education of the patient about his/her one or more pathologies and treatments; the digitization of administrative and/or medical documents of the patient by means of a digital “safe” system; the scheduling of teleconsultations or in-person consultations with any healthcare professional; the secure message exchange with all the healthcare professionals, be them in a hospital or outside the hospital, such as ambulatory care facilities, that are in charge of said patient; a frequently asked questions forum moderated by healthcare professionals; a chatbot, or intelligent digital assistant based on by machine-learning, to answer patients' questions; a medical calendar offering an overview of all the events in the healthcare path, such as blood tests, taken medications, performed medical exams, said medical calendar being filled in the patient himself, by the healthcare professionals in charge of him, as well as automatically.

These features on the patient-side of the present system, further comprise technical means such as a module configured to generate medication reminders, said reminders being generated based on the patient's pathologies and/or the administered treatments or by means of an artificial intelligence based on prescribed treatments. The medication reminder module further allows visualization and validation by healthcare professionals, in order to avoid patient non adherence.

In addition, the system comprises a secure sharing module allowing to access to a patient's data, said module enables the patient and one or more authorized healthcare professionals to share all the patient's data with another healthcare team in a hospital in France or abroad. The sharing module further comprises an automated translation system.

Furthermore, the telehealth system of the present invention comprises features related to the practical organization of the daily life of the healthcare professionals.

On the health provider-side of the present system, these features comprise:
a dashboard for displaying a summary of the tasks to be done or the tasks in progress; alerts related to a health state of the patients the health provider is in charge of, said alerts being customizable by means of criteria specific to the specialty of said health provider; recommendations related to a specific patient; automatically generated prescriptions; electronic medical records of the patients; a digital calendar; instant messages exchanged with their patients and/or colleagues by means of a secure, encryption-based, messaging module;
an integrated teleconsultation module comprising a payment system; said teleconsultation module further comprising a speech recognition and recording module capable of generating a text report based on the recorded voice;
a tele-expertise module in order to send requests to one or more colleagues;
a module for managing information concerning patients' illnesses or treatments.

Advantageously, the digitalization and automation of multiple tasks performed by health professionals and the direct link between health professionals and colleagues and/or patients ease the exchange of information and the communication, thereby reducing the workload and increase the productivity of health professionals.

Examples of said links between health professionals and colleagues and/or patients according to one embodiment of the present invention are illustrated in FIG. 2.

These features on the healthcare professional side of the present system further comprise technical means such as a prescription module configured to automatically generate or suggest medical prescriptions mentioning treatments and appropriate doses thereof, said module being capable of generating prescriptions in different formats by means of algorithms based on: the patient's pathologies and/or the administered treatments, the data from the patient's shared medical record, the results of questionnaires the data collected from connected objects.

This prescription module is also available to pharmacies. On the pharmacy side, the prescription module notifies about the need to make a drug purchase order when a prescription is generated, or it automatically generate said purchase order.

The present system is capable of interpreting written, digital or oral prescriptions thanks to an artificial intelligence which converts human writing or oral voice into a digital format and it further allows physicians to make electronic prescriptions by inserting the prescription directly in digital form, in a dedicated space of the system.

Furthermore, the present system allows to reorganize the institutions delivering care, such as hospitals, clinics, nursing homes or doctors' offices, so that the hospital services, especially emergency services, in order to plan the admissions and discharges of future hospitalizations in advance.

The present system can be interoperable with other medical platforms providing other functions in medical institutions in France, in particular the DMP, or internationally, such as Cerner or Epic in the United States.

The general purpose of the present system is to develop artificial intelligences on the basis of the totality of collected data, in order to improve the understanding of social sciences, by means of a secure system and/or a blockchain-based technology.

While various embodiments have been described, the detailed description is not to be construed as being limited hereto. Various modifications can be made to the embodiments by those skilled in the art without departing from the true spirit and scope of the disclosure as defined by the claims.

### DESCRIPTION OF THE DRAWINGS

FIG. 1 is a flowchart illustrating the telehealth system of the present invention according to a first embodiment.

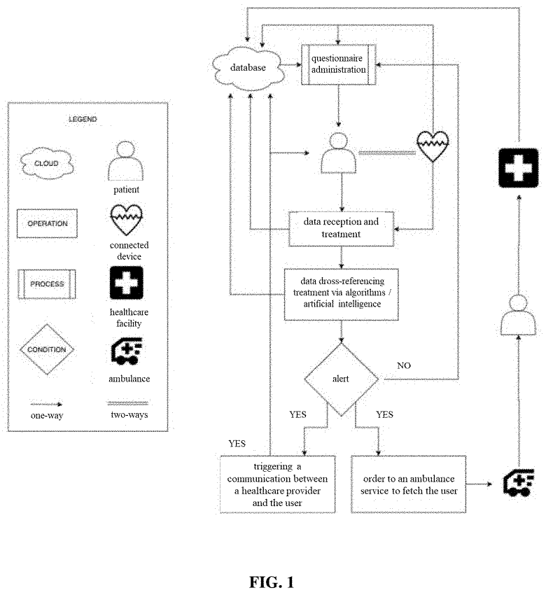

FIG. 2 is a flowchart illustrating the telehealth system of the present invention according to a second embodiment.

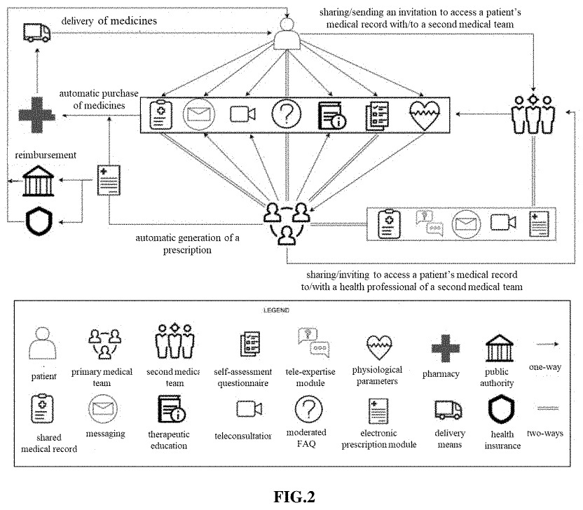

1-16. (canceled)

17. A telehealth system for rescuing a user in need thereof, the system comprising a central server, a transmission system and at least one peripheral device configured to continuously or intermittently transmit health-related user's data to the central server via the transmission system; the health-related user's data comprising at least one measurement of a physiological parameter; and the central server comprising:
a module configured to receive the health-related user's data;
a memory configured to store:
the health-related user's data;
a user's alert list comprising a plurality of recipient devices;
a library of instructions comprising: sending a communication request to all the recipient devices in the user's alert list; triggering a call to an emergency service, ordering an assistance service to fetch the user; and
a library of physiological parameters wherein a reference value or a reference range of values is associated with each physiological parameter;
a processing unit configured to:
compare the at least one measurement of a physiological parameter with the reference value or the reference range of values associated with said physiological parameter in the library;
determine at least one risk index based on the comparison;
determine a level for the at least one risk index, the risk index level being selected from a group consisting of: low, moderate, high;
based on the risk index level, execute at least one instruction from the list of instructions, and for a moderate or a high risk level, the at least one instruction comprises automatically sending a communication request to all the recipient devices in the user's alert list and, in case of no response within a predetermined interval of time Δt, a call to an emergency service or an order to an assistance service to fetch the user.

18. The telehealth system according to claim 17, wherein the plurality of recipient devices in the user's alert list comprises:
at least one user-side communication device;
at least one communication device of a second subject designated by the user;
the communication devices that respond to a vicinity criterion.

19. The telehealth system according to claim 18, wherein the vicinity criterion is:
being connected to the same wireless network as the at least one user's peripheral device or the at least one user-side communication device; and/or
being located within a predetermined distance from the user-side communication device at least one peripheral device, preferentially the predetermined distance is ranging between 0 meters and 500 meters.

20. The telehealth system according to claim 17, wherein the health-related user's data further comprise:
an electronic medical record of the user and/or
the user's answers to the questions of a questionnaire, the questionnaire comprising questions that are neutral to the user and questions that are generated by means of an algorithm based on the health-related user's data;
and wherein the determination of at least one risk index is further based on the user's data in the electronic medical record and/or the user's answers to the questions of the questionnaire.

21. The telehealth system according to claim 20, wherein the electronic medical record comprises genetic factors and/or epidemiologic factors.

22. The telehealth system according to claim 17, wherein the physiological parameter is selected from a group comprising: oxygen saturation, respiratory rate, blood pressure, heart rate, temperature, weight, blood pressure, respiratory rate, blood oxygen saturation, pulse rate, heart rate variability, stroke volume, cardiac output, cardiac index, pulse pressure, systemic vascular resistance, ECG, mean arterial pressure, sweat level, skin temperature, body temperature, glucose level and wherein the risk index is selected from a group comprising: anaphylactic shock, stroke, heart failure, cardiovascular diseases, respiratory deficiency, sleep apnea, endocrinal disease, metabolic disease, infectious or parasitic diseases, digestive diseases, immune system diseases, nephrological disorders, sexually transmitted diseases, viral diseases, smoking addiction, drug use addiction, arterial hypertension, cancer, kidney diseases, gynecological diseases, neurodegenerative diseases, neurological diseases, psychiatric diseases, post-operative risk, circulatory system disease, sudden cardiac death, nutritional diseases or a risk of sudden death.

23. The telehealth system according to claim 17, wherein the processing unit is further configured to:
in case of response to the communication request, classify said response as a rescue request response or a non-rescue request response; and
for a rescue-request response, automatically trigger a call to an emergency service or an order to an ambulance service to fetch the user;
for a non-rescue-request response, automatically send a second communication request to all the recipients in the user's alert list.

24. The telehealth system according to claim 17, wherein the predetermined interval of time Δt ranges between 10 and 60 seconds and wherein the predetermined interval of time Δt for a moderate risk index level is superior to the interval of time Δt for a high risk index level.

25. The telehealth system according to claim 17, wherein the list of instructions further comprises:
sending an alert to a subset of devices selected from the user's alert list;
generating a questionnaire;
generating a medicine prescription;
generating a medicine purchase order for the prescribed medicine;
automatically delivering the prescribed medicine;
generating a pre-medical evaluation report;
generating a post-medical evaluation report;
scheduling a user's consultation with a healthcare provider;
sending a medication reminder to the user;
sending an alert to a healthcare provider.

26. A computer-implemented method for rescuing a user in need thereof, the method comprising the following steps:
a) measuring and/or collecting health-related data of a user via at least one peripheral device, the health-related data comprising at least one measurement of a physiological parameter;
b) transmitting the health-related data from the peripheral device to a central server via a transmission system, the central server comprising a memory;
c) storing, preferentially using a distributed ledger or blockchain technology, the health-related data in the memory of the central server;
d) generating a user's alert list comprising a plurality of recipient devices;
e) continuously or intermittently comparing the at least one measurement of a physiological parameter with a reference value or a reference range of values for said physiological parameter stored in a library;
f) determining at least one risk index based on the result of the comparison step e) and on the user's health-related data;
g) determining a level for the at least one risk index, the risk index level being selected from a group consisting of: low, moderate, high; based on the at least one risk index and on the risk index level, selecting at least one instruction from a library of instructions;
wherein, for a moderate or high risk, the selected at least one instruction comprises automatically sending a communication request to all the recipient devices in the user's alert list and, in case of no response to the communication request within a predetermined interval of time Δt, a call to an emergency service or an order to an assistance service to fetch the user at a time.

27. The method according to claim 26, further comprising a step of
b.2) evaluating a quality metrics of the at least one measurement of a physiological parameter by means of an artifact detection algorithm;
and the step f) of determining at least one risk index is further based on the evaluated quality metrics.

28. The method according to claim 26, wherein the step of generating a user's alert list comprises:
receiving as input information regarding at least one user-side communication device and at least one communication device of a second subject designated by the user;
identifying neighbor devices based on a user's vicinity criterion; the preferred vicinity criterion being: identifying the communicating devices that are connected to the same wireless network as the at least one user's peripheral device or the at least one user-side communication device.
automatically generating a user's alert list comprising, as recipients: the at least one user-side communication device; the at least one communication device of a second subject designated by the user and the identified neighbor devices.

29. The method according to claim 26, further comprising:
repeating the steps a) to c) so as to obtain health-related data from a plurality of users;
building, by means of a machine learning algorithm, a predictive model based on the data from a plurality of users.

30. The method according to claim 26, further comprising a step of inputting the health-related data of a user into the predictive model and obtaining as output a value predictive of a future clinical event of the user.

31. A computer program product for rescuing a user in need thereof, the computer program product comprising instructions which, when the program is executed by a computer, cause the computer to carry out the steps of the method according to claim 26.

32. A computer-readable storage medium comprising instructions which, when the program is executed by a computer, cause the computer to carry out the steps of the method according to claim 26.
